(19) United States
(12) Patent Application Publication
(10) Pub. No.: US 2026/0364598 A1
(43) Pub. Date: Nov. 19, 2026

(54) BIOCHIP AND BIOCHIP COMPENSATION SYSTEM

(71) Applicant: Cepheid, Sunnyvale, CA (US)

(72) Inventors: Arun MANICKAM, San Jose, CA (US); Sundarasiva Rao GIDUTURI, Milpitas, CA (US)

(73) Assignee: Cepheid, Sunnyvale, CA (US)

(21) Appl. No.: 19/657,790

(22) Filed: Apr. 24, 2026

Related U.S. Application Data
(60) Provisional application No. 63/795,030, filed on Apr. 25, 2025.

(51) Int. Cl. B01L 3/00

(52) U.S. Cl. CPC B01L 3/502715; B01L 2200/147; B01L 2300/0663; B01L 2300/0819; B01L 2300/1827

(57) ABSTRACT

A biochip includes a biosensor configured to detect target analytes, a temperature sensor configured to provide temperature data for compensating the biosensor, a processing unit configured to process signals from the biosensor and the temperature sensor, and a memory configured to store calibration data, trim coefficients, or both.

### CROSS-REFERENCE TO RELATED APPLICATIONS

This application claims the benefit of and priority to U.S. Provisional Application No. 63/795,030, filed on Apr. 25, 2025, titled BIOCHIP AND BIOCHIP COMPENSATION SYSTEM, the disclosure of which is hereby incorporated by reference in its entirety.

### BACKGROUND

Biochips are microelectronic devices integrating one or multiple laboratory functions on a single chip. Biochip technology offers a compact and efficient platform for detecting and analyzing biological information. By miniaturizing and automating laboratory processes, biochips dramatically reduce reagent consumption, decrease sample requirements, and increase the speed and throughput of tests.

Compensation systems are designed to adjust and standardize the performance of the biochip to ensure precision in test results. The compensation system ensures that the biochip and any associated laboratory instruments operate within the desired parameters, thereby enhancing reproducibility and consistency in test results.

### SUMMARY

In general terms, this disclosure is directed to a biochip and to a biochip compensation system. In a non-limiting example, this disclosure relates to a biochip integrated with a temperature sensor and a memory, as well as a method for compensating the biochip.

One aspect of the present disclosure pertains to a biochip comprising: a biosensor configured to detect target analytes; a temperature sensor configured to provide temperature data for compensating the biosensor; a processing unit configured to process signals from the biosensor and the temperature sensor; and a memory configured to store calibration data, trim coefficients, or both.

Another aspect of the present disclosure pertains to a system configured to perform compensation, the system comprising: a wafer tester configured to interface with a wafer and execute a compensation process; a heater configured to regulate the temperature of the wafer during the compensation process; a chip interface configured to facilitate data communication with a biochip for obtaining a temperature reading of the biochip, wherein the biochip includes a temperature sensor configured to provide temperature data for compensation, and a memory configured to store calibration data, trim coefficients, or both, including a temperature-based correction factor; and a controller configured to manage the operation of the heater, and determine the temperature-based correction factor.

A further aspect of the present disclosure pertains to a method for calculating and storing calibration data, trimming coefficients, or both in biochips of a wafer, the method comprising: receiving temperature data from a temperature sensor of the biochips during a compensation process; regulating the temperature of the wafer by controlling a heater based on the temperature data to achieve a predetermined condition; measuring operating parameters of the biochips under the predetermined condition; calculating calibration data, trim coefficients, or both, including temperature-based correction data, based on the measured operational parameters and the temperature data; and storing the calibration data, trim coefficients, or both in a memory of each biochip.

Yet another aspect of the present disclosure pertains to a chip carrier device comprising: a frame; an access point positioned at an end of the frame; a fluidic channel extending at least partially through the frame and extending from the access point to a chip receiving region; and the chip receiving region formed in the frame; a biochip arranged in the chip receiving region, the biochip comprising: a biosensor configured to detect target analytes, a temperature sensor configured to provide temperature data to compensate the biosensor, a processing unit configured to process signals from the biosensor and the temperature sensor, and a memory configured to store calibration data, trim coefficients, or both, including temperature-based correction factors.

Still another aspect of the present disclosure pertains to a sample cartridge assembly comprising: a sample cartridge; and a chip carrier device having: a frame; an access point arranged at an end of the molded frame; at least one fluidic channel extending at least partially through the molded frame and extending from the access point to the chip receiving region; and a chip receiving region formed in the frame, the chip receiving region being configured to accommodate a biochip, wherein the biochip includes: a biosensor configured to detect target analytes, a temperature sensor configured to provide temperature data to compensate the biosensor, a processing unit configured to process signals from the biosensor and the temperature sensor, and a memory configured to store calibration data, trim coefficients, or both, including temperature-based correction factors.

Another aspect of the present disclosure pertains to a method of using calibration data in a biochip system, the method comprising: receiving compensation data from a biochip, wherein the biochip includes: a biosensor detecting target analytes, a temperature sensor monitoring an operating temperature of the biochip, a processing unit processing signals from the biosensor and the temperature sensor, and a memory storing calibration data, trim coefficients, or both, including a temperature-based correction factor; applying the calibration data, the trim coefficients, or both to adjust operational parameters of the temperature sensor in the biochip; detecting the presence of the target analytes to conduct a measurement; processing and correcting the measurement results by applying the calibration data, the trim coefficients, or both; and outputting the processed and corrected measurement results for further analysis.

Still another aspect of the present disclosure pertains to a biochip comprising: a biosensor configured to detect target analytes; a temperature sensor configured to provide temperature data for compensating the biosensor; a processing unit configured to process signals from the biosensor and the temperature sensor; and a memory configured to store calibration data, trim coefficients, or both, wherein at least one of the biosensor, the temperature sensor, the processing unit, and the memory is integrated on a single chip.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

FIG. 1 is a flow diagram illustrating the manufacturing and testing process of a biochip across different facilities.

FIG. 2 is a schematic diagram illustrating an example instrument.

FIG. 3 is a perspective view of an example cartridge.

FIG. 4 is a perspective view of an example module.

FIG. 5 is an exploded view of an example cartridge.

FIG. 6 is a block diagram illustrating an example biochip.

FIG. 7 is a schematic diagram illustrating an example biochip.

FIG. 8 is a block diagram illustrating a compensation system.

FIG. 9 is a flow chart illustrating an example method of compensating a biochip.

FIG. 10 is a block diagram illustrating an example testing system that includes a sample cartridge and a laboratory instrument.

FIG. 11 is a flow chart illustrating an example process for performing measurements using a biochip within an instrument.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

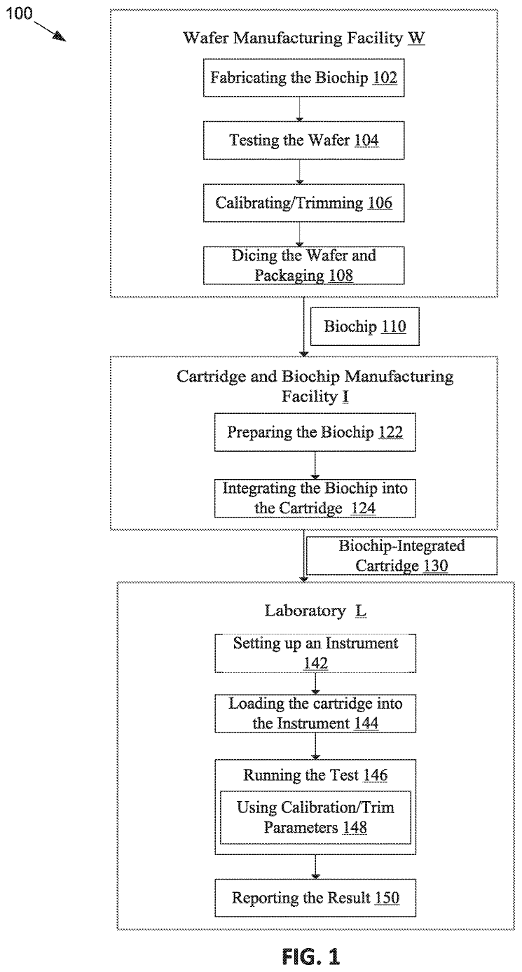

FIG. 1 is a flow diagram illustrating an example system 100 for manufacturing, testing, and using a biochip across different locations. The example process is divided into three main stages: a Wafer Manufacturing Facility W, a Cartridge and Biochip Integrating Facility I, and a Laboratory L. The example Wafer Manufacturing Facility W performs operations 102, 104, 106, and 108. The example Cartridge and Biochip Manufacturing Facility I performs operations 122 and 124. The example laboratory L performs operations 142, 144, 146, 148 and 150. Other embodiments include more, fewer, or different operations from the example illustrated in FIG. 1.

A biochip 110 is fabricated in the Wafer Manufacturing Facility W before integration. As illustrated in FIG. 1, the operation 102 is performed to fabricate the biochip 110. In some embodiments, a temperature sensor is integrated into the biochip 110 to monitor and control thermal conditions, which affect the performance of the biochip 110. In some embodiments, a memory is integrated into the biochip 110 to store operational data and instructions necessary for its function. In some embodiments, multiple electrical contacts are deposited onto the biochip 110, configured to interface with an external electrical device.

Subsequently, the operation 104 tests the wafer to ensure quality and functionality. In some embodiments, the operation 104 is performed to analyze the overall performance of the wafer, such as evaluating its electrical characteristics, and structural integrity. For example, the defective or malfunctioning chips will be identified before cutting into individual chips.

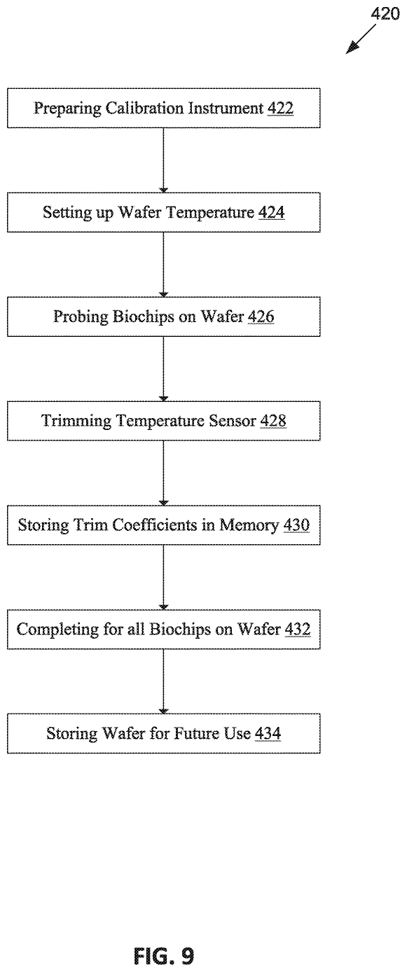

Further, the operation 106 is performed to compensate the biochip at wafer-level. In some embodiments, the compensation includes calibrating the biochip, trimming the biochip, or a combination thereof, to ensure that the biochip 110 functions accurately under varying environmental conditions. The compensation process in operation 106 not only addresses temperature variations but also performs trimming to fine-tune circuit or sensor parameters, correct manufacturing variations and optimize performance for future measurements. Further, the calibration data or trim coefficients, including temperature compensation parameters and sensor calibration coefficients, are stored in a memory to facilitate real-time adjustments and maintain optimal performance during operation. An example of operation 106 is illustrated and described in detail with reference to FIG. 9.

Finally, the operation 108 is performed to dice the wafer into individual chips, also known as a die. In some embodiment, the operation 104 is performed to separate the wafer into individual chips, each including a functional circuit. Further, the individual chips will be packaged before integrated into a system.

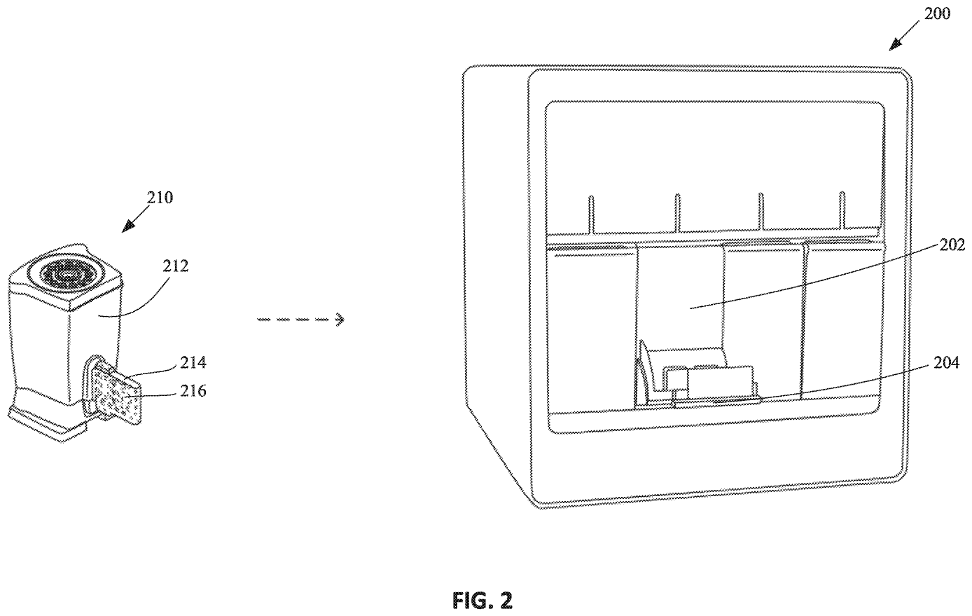

The biochip-integrated cartridge 130 is manufactured for laboratory testing in the Cartridge and Biochip Manufacturing Facility I. As illustrated in FIG. 1, the operation 122 is performed to prepare the biochip 110 for integration into a cartridge (shown in FIG. 2). In some embodiments, quality control is performed at operation 122 to ensure the biochip 110 is functional before it is assembled into the cartridge.

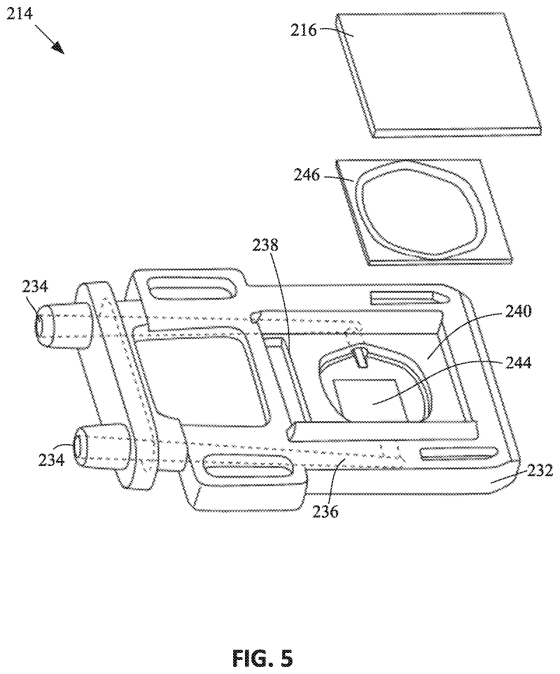

Further, the operation 124 is performed to integrate the biochip 110 into a chip carrier of the cartridge (shown in FIG. 5). In some embodiments, the operation 124 ensures that the biochip 110 is mounted within the cartridge to establish alignment, electrical connectivity, and mechanical stability.

In Laboratory L, testing is conducted, and results are reported. As illustrated in FIG. 1, the operation 142 is performed to set up an instrument in preparation for testing. Subsequently, the operation 144 is performed to load the cartridge into the instrument that will be used for testing in the laboratory. In some embodiments, the operation 144 ensures that the cartridge is positioned within the instrument to establish electrical and mechanical connections required for testing.

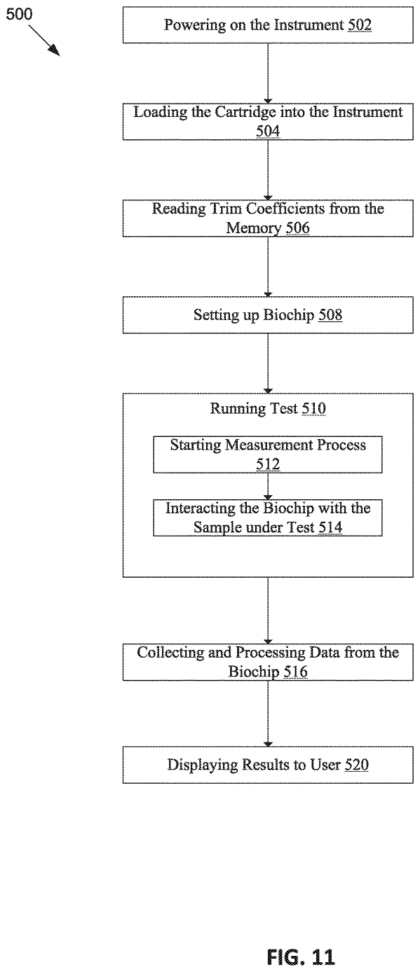

Further, the operation 146 is performed to run the test of a biological sample that is analyzed by the biochip 110. As part of operation 146, a sub-step 148 is executed where trim coefficients are applied to ensure accurate and reliable test results. The parameters help compensate for variations in sensor performance, temperature fluctuations, and manufacturing tolerances, thereby enhancing the precision and reproducibility of the biochip's analysis. An example of operation 146 is illustrated and described in further detail with reference to FIG. 11.

Finally, the operation 150 is performed to process and report the results. An example of operation 150 is illustrated and described in further detail with reference to FIG. 11.

Performing compensation of the temperature sensors in the biochips at the wafer level or inspection stage, prior to instrument loading, provides multiple advantages, including early defect detection that improves yield, reduces waste, and lowers production costs. In some embodiments, compensating the temperature sensor of the biochip includes calibrating the temperature sensors, trimming sensor parameters, or a combination thereof. Calibration ensures that the sensors meet specified accuracy requirements, while trimming corrects for manufacturing variations and enhances long-term stability. Conducting these compensation processes at an early manufacturing stage improves the reliability of the biochips for critical applications and simplifies the final instrument design by reducing the need for complex system-level calibration. Further, integrating inspection and compensation into wafer-level manufacturing streamlines production, facilitates automation and scalability, and promotes consistent performance across devices and production batches.

FIG. 2 is a schematic diagram illustrating an example instrument 200. The example instrument 200 includes a receiving module 202 and a module door 204. As shown in FIG. 2, an example sample cartridge assembly 210 is also illustrated. The sample cartridge assembly 210 includes a sample cartridge 212 and a chip carrier device 214. The chip carrier device 214 further includes a biochip 216.

The instrument 200, in cooperation with the sample cartridge assembly 210, measures the properties of a biological sample. In some embodiments, the instrument 200 performs analytical tests, such as may be performed in a laboratory. In some examples, the instrument 200 conducts different assays to detect diseases, identify the presence of specific genetic material, or analyze biomarkers. In some embodiments, the instrument 200 operates to receive the sample cartridge assembly 210. As an example illustrated in FIG. 2, the instrument includes a module door 204 that can be opened to allow insertion of the sample cartridge 212 into the receiving module 202. In some embodiments, the instrument 200 includes more than one (e.g., a plurality of) receiving module 202, each with its corresponding module door 204. In other examples, the receiving module 202 is also responsible for supplying power and managing the fluidics within the sample cartridge 212 and the biochip 216.

The receiving module 202 receives and interfaces with the sample cartridge 212. In some embodiments, the receiving module 202 acts as a bridge between the sample cartridge 212 and the instrument 200, enabling power delivery, data communication, optical or electrical sensing, and fluidic control for sample testing. For example, the receiving module 202 includes multiple electrical contacts and one or more detection units. In some embodiments, the electrical contacts engage with the biochip 216, allowing the instrument 200 to power, control, and communicate with the biochip for data acquisition. In other embodiments, the receiving module 202 includes an optical detection unit to detect fluorescence. In other examples, the receiving module 202 is also responsible for supplying power and managing the fluidics within the sample cartridge 212 and the biochip 216, ensuring controlled reagent flow and continuous sample processing throughout the analysis.

The module door 204 provides access to the receiving module 202, allowing the sample cartridge 212 to be inserted or removed from the instrument 200. In some embodiments, the module door 204 moves between open and closed positions, such as by pivoting or swinging on a hinge. For example, when the module door 204 is open, the sample cartridge 212 is allowed to be inserted into the receiving module 202. Once the sample cartridge 212 is loaded, the module door 204 pivots to a closed position, enabling the instrument 200 to start processing the sample. After the analysis is complete, the module door 204 pivots open again, allowing the sample cartridge 212 to be removed.

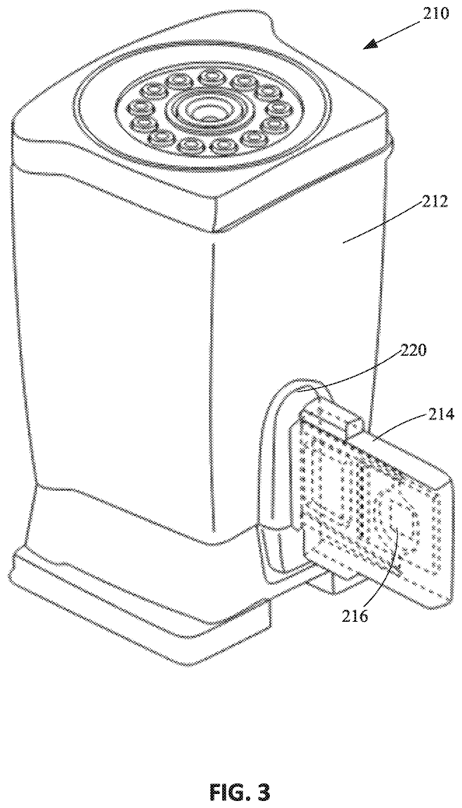

An example of the sample cartridge assembly 210 is illustrated and described in further detail with reference to FIG. 3.

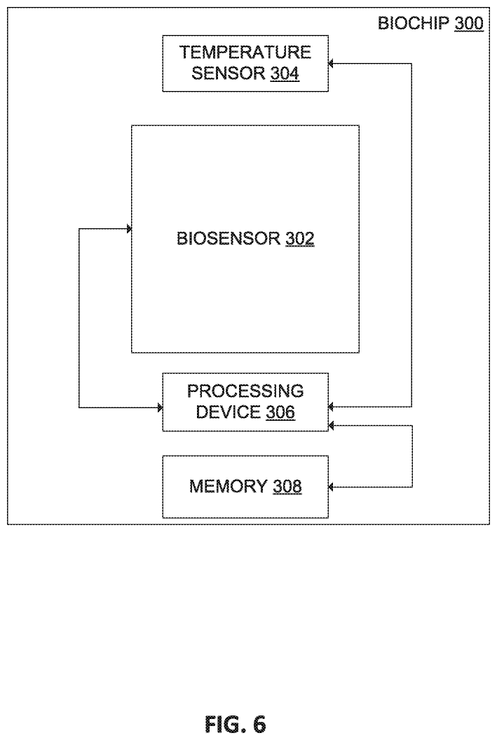

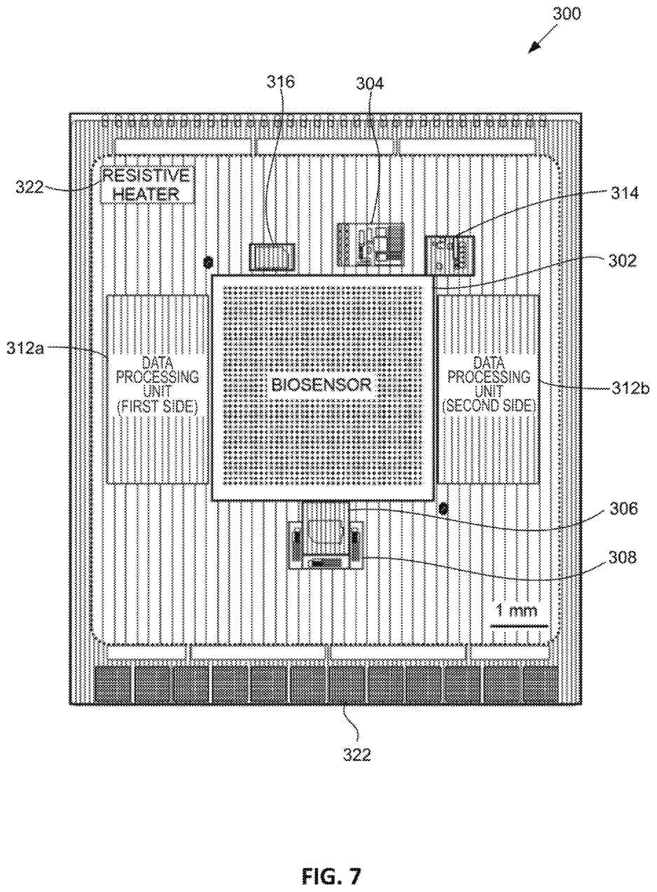

An example of the biochip 216 is illustrated and described in further detail with reference to FIGS. 6 and 7.

FIG. 3 is a perspective view of an example sample cartridge assembly 210. The sample cartridge assembly 210 includes a sample cartridge 212, a chip carrier device 214, and a chip carrier receptacle 220. In some embodiments, the chip carrier device 214 further includes a biochip 216.

The sample cartridge assembly 210 includes components for receiving and processing a biological sample for analysis by the instrument 200. In some embodiments, the sample cartridge assembly 210 is configured to facilitate the introduction, handling, and processing of a biological sample for analysis by the instrument 200. For example, the sample cartridge assembly 210 interfaces with the instrument 200 to ensure the sample reacts with reagents and is analyzed by the instrument 200.

The sample cartridge 212 is configured to receive the biological sample and hold the chip carrier device 214. In some embodiments, the sample cartridge 212 includes one or more processing chambers for sample handling and processing. In some examples, the processing chambers are configured to facilitate biochemical, molecular, or analytical reactions for sample preparation and testing. For example, the biological sample is combined with reagents, buffers, or enzymes in the processing chambers and is reacted to generate analytical results. In some embodiments, once the biological sample is added to the sample cartridge 212, the sample cartridge assembly 210 is loaded into the receiving module 202 for processing and analysis.

The chip carrier device 214 is configured to secure the biochip 216 while ensuring proper integration with the receiving module 202 (shown in FIG. 2). In some embodiments, the chip carrier device 214 facilitates alignment of the biochip 216 with the electrical contacts or the detection units within the receiving module 202. In some examples, the chip carrier device 214 establishes a fluidic connection between the biochip 216 and the sample cartridge 212. For example, the chip carrier device 214 includes one or more fluidic channels 236 (shown in FIG. 5) that transport the biological sample from the sample cartridge 212 to the biochip 216 for processing and analysis.

The chip carrier receptacle 220 is a slot formed on a sidewall of the sample cartridge 212. In some embodiments, the chip carrier receptacle 220 receives and secures the chip carrier device 214. For example, the chip carrier receptacle 220 secures the chip carrier device, ensuring that the biochip 216 is fluidically connected to the sample cartridge 212. In some embodiments, the chip carrier receptacle 220 secures the chip carrier device 214 while keeping the biochip 216 accessible to the receiving module 202.

An example of the biochip 216 is illustrated and described in further detail with reference to FIGS. 6 and 7.

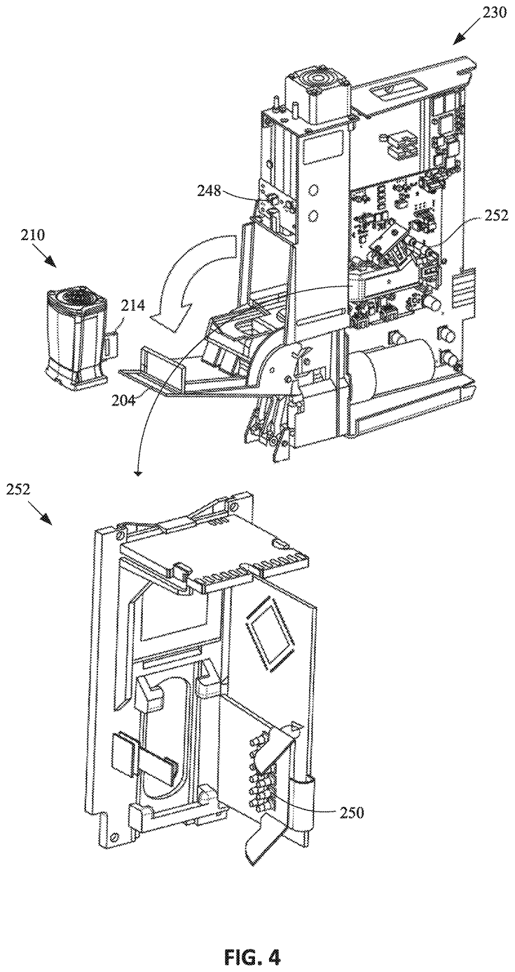

FIG. 4 is a perspective view of an example module 230. The example module 230 includes a module door 204, a temperature control system 248, and an instrument interface 252. In some embodiments, the instrument interface 252 includes a plurality of pogo pins 250. As shown in FIG. 4, a sample cartridge assembly 210 is also illustrated. As previously discussed, the cartridge includes a chip carrier device 214.

The module door 204 provides access to the sample cartridge assembly 210, as previously discussed. In some embodiments, the module door 204 moves between open and closed positions, such as by pivoting or swinging on a hinge. As an example illustrated in FIG. 4, the module door 204 is in an open position that allows the sample cartridge assembly 210 to be inserted in or moved away.

The temperature control system 248 is designed to regulate and maintain specific temperature conditions required for the testing process. In some embodiments, the temperature control system 248 ensures that temperature-sensitive reactions, measurements, or sample preparations occur under optimal thermal conditions. For example, the biochip of the chip carrier device 214 is maintained at a suitable temperature to facilitate proper biochemical reactions and readings, enhancing the accuracy of the results generated by the instrument. In some examples, the temperature control system 248 includes a combination of sensors, heating and cooling elements, a control unit, and insulation mechanisms, all working together to achieve and sustain the desired temperature range.

The instrument interface 252 of the example module 230 is incorporated into the module 230 within a bay where the sample cartridge assembly 210 is received. In some embodiments, the instrument interface 252 includes pogo pins 250 that engage with corresponding contacts on the biochip. In some examples, the instrument interface 252 reads calibration data or trimming coefficients from the biochip. For example, reading the calibration data or trimming coefficients allows the instrument to automatically adjust its settings and algorithms according to the specific characteristics of the biochip.

The pogo pins 250 are electrical contacts. In some embodiments, the pogo pins 250 are configured for interacting with electrical contacts of the biochip. In some embodiments, when the chip carrier device 214 is inserted into the module 230, the pogo pins 250 engage with corresponding contacts of the biochip to allow the module 230 to control analysis of the biological sample within the biochip.

FIG. 5 is an exploded view of an example chip carrier device 214. In this example, the chip carrier device 214 includes a gasket 246 and a frame 232. The frame 232 further includes an access point 234, at least one fluidic channel 236, a chip receiving region 240, and a target region 244.

The gasket 246 is a mechanical seal that prevents leakage between two or more surfaces. In some embodiments, the gasket 246 provides a fluid-tight seal between the frame 232 and the biochip 216. In some examples, the gasket 246 is coupled to the frame 232 to maintain alignment and prevent leakage during operation. In some embodiments, the gasket 246 is a rectangular webbing structure designed to fit within the chip receiving region 240.

In some embodiments, the gasket 246 includes tabs to facilitate handling and placement, allowing a placement tool to position it within the frame 232. In some examples, the gasket 246 also incorporates a compression ring at its center, enhancing the seal between the biochip 216 and the frame 232 by applying uniform pressure across the interface.

The frame 232 provides structural support and secures the biochip 216 in place. In some embodiments, the frame 232 is a frame formed using an injection molding process. In some examples, the frame 232 includes alignment features, such as guide rails, recesses, cavities, or interlocking mechanisms, to ensure the biochip 216 is correctly positioned and secured. In some embodiments, the frame 232 incorporates the gasket 246 to prevent contamination and maintain fluid integrity during sample processing. In some examples, the frame 232 includes an integrated fluidic pathway, such as the fluidic channel 236 that directs the flow of biological samples, reagents, or buffer solutions to designated regions on the biochip 216.

The frame 232 includes an access point 234 that are positioned at an end of the frame 232. In some embodiments, the frame 232 includes two access points 234 that are configured to input and output a biological sample. For example, the two access points 234 are fluidly connected to the sample cartridge 212. In some examples, one of the two access points 234 directs the biological sample to the fluidic channel 236, and one of the two access points 234 directs the biological sample from the fluidic channel 236 to the sample cartridge 212, forming a path through the chip carrier device 214.

The fluidic channel 236 defines a fluidic pathway for transporting a biological sample. In some embodiments, the fluidic channel 236 is integrated into the frame 232, where one or more fluidic channels 236 can be formed to facilitate the biological sample movement. For example, the fluidic channel directs the biological sample from the access point 234 to the biochip 216.

In some embodiments, multiple fluidic channels 236 are present to accommodate different processing steps, such as reagent mixing, washing, or waste disposal. In some examples, the fluidic channel 236 incorporates microfluidic design elements, such as capillary forces, valves, or pumps, to regulate sample flow and optimize interaction with the biochip 216.

The chip receiving region 240 is configured to receive the biochip 216. In some embodiments, the chip receiving region 240 aligns the biochip 216 with the fluidic channel 236. In some examples, the chip receiving region 240 is formed within the frame 232. For example, the chip receiving region 240 is a recessed area within the frame 232, designed to accommodate the biochip 216.

The target region 244 is a designated area of the chip carrier device 214. In some embodiments, the target region 244 is designed to facilitate the analysis of samples directly on the biochip 216. In some examples, the target region 244 acts as a transparent window that allows specific wavelengths of light to penetrate for activating fluorescent tags used in various assays. This targeted illumination enables the photodetectors of the biochip 216 to detect the presence of fluorescence, thereby allowing the instrument to accurately process and interpret the analytical data. Therefore, the target region 244 exposes the biochip 216 to the necessary light conditions required for the fluorescence-based tests.

FIG. 6 is a block diagram illustrating an example biochip 300. In the example illustrated in FIG. 6, the biochip 300 includes a biosensor 302, a temperature sensor 304, a processing unit 306, and a memory 308.

The biochip 300 is a microelectronic device that analyzes and detects biological materials. The biochip 300 hosts biomedical reactions to screen biological analytes or test for biological analytes.

The biosensor 302 is a component that detects biological analytes. The biosensor 302 combines a biological sensing element with a transducer to detect and measure a biological, chemical, or physical process. In some embodiments, the biosensor 302 includes a biosensor array.

The temperature sensor 304 is a component that monitors the operating temperature of the biochip 300. In some embodiments, the temperature sensor 304 is positioned adjacent to the biosensor 302 to provide temperature data to compensate the biosensor 302.

The processing unit 306 manages and processes data received from other components of the biochip 300. In some embodiments, the processing unit 306 is configured to process signals from the biosensor 302 and the temperature sensor 304. For example, the processing unit 306 includes a digital core to digitize the analog signals.

The memory 308 is a component configured to store data related to the operation of the biochip 300. For example, the memory 308 stores the data generated by other components, such as the temperature sensor 304 and the biosensor 302. In some embodiments, the memory 308 stores calibration data or trimming coefficients or both, including temperature-based correction factors.

In the example illustrated in FIG. 6, the biosensor 302, the temperature sensor 304, and the memory 308 are each connected to the processing unit 306, forming an integrated system.

FIG. 7 is a schematic diagram illustrating another example of the biochip 300. In the example illustrated in FIG. 7, the biochip 300 includes a biosensor 302, a temperature sensor 304, a processing unit 306 and a memory 308, which are the same or similar to those illustrated and described with reference to FIG. 6. The example biochip 300 shown in FIG. 7 further includes a first data processing unit 312a, a second data processing unit 312b, a reference generator 314, a test control 316, a resistive heater 318, and a plurality of I/O pads 322. Other examples of the biochip 300 can include any one or more of the components illustrated in FIG. 7, and combinations thereof. In other words, other examples may have more or fewer components than shown in FIG. 7.

As previously discussed, the biochip 300 is a microelectronic device. In some embodiments, the microelectronic device is fabricated using semiconductor fabrication techniques, such as Complementary Metal-Oxide-Semiconductor (CMOS) technology that integrates microelectronic components with biochemical functionalities. For example, the biosensor 302, the temperature sensor 304, the processing unit 306, and the memory 308 are integrated on a single chip by using the CMOS technology. In some examples, the biochip 300 interfaces with lab instruments for signal detection and data acquisition. Further, the biochip 300 processes biological or chemical signals and transmits the processed data to the lab instrument.

The biosensor 302 includes a plurality of individual sensor elements. In some embodiments, the plurality of individual sensor elements is arranged in a matrix including a plurality of rows and columns. In the illustrated example, the plurality of individual sensor elements is arranged in a 32×32 matrix including 1024 individual sensor elements. In other embodiments, the biosensor 302 has other quantities of sensor elements, including more or fewer sensor elements. In other embodiments, the biosensor 302 has sensor elements that are arranged in other configurations or orientations (e.g., triangle, diamond, circle, or the like).

In some embodiments, the biosensor 302 is designed to detect specific biological interactions, such as utilizing techniques of Continuous Wave Fluorescence (CWF), Time-Gated Fluorescence (TGF), a combination of CWF and TGF, or other detection techniques. In some examples, the biosensor 302 seamlessly interfaces with lab instruments to provide rapid and accurate analysis of biological samples. As shown in the example in FIG. 7, the biosensor 302 further converts the detected data into electrical signals and transmits the signals to the processing unit 306 to be processed through algorithms to extract the needed information.

As previously discussed, the temperature sensor 304 monitors the operating temperature of the biochip 300. In some embodiments, the temperature sensor 304 is used for compensating the biosensor of the biochip to ensure reliable biochemical measurements. In some embodiments, the temperature sensor 304 includes an improved architecture with a one-point trim at room temperature, allowing for precise compensation at the wafer level or during inspection before instrument loading. In some embodiments, the temperature sensor 304 connects to the processing unit 306, and the temperature reading is processed by the processing unit 306. In some examples, the temperature sensor 304 is positioned proximal to the biosensor 302 to facilitate real-time adjustments and compensations to the readings of the biosensor 302. For example, by placing the temperature sensor 304 close to the biosensor 302, the lag time is minimized between detecting a temperature change and adjusting the response from the biosensor 302.

As previously discussed, the processing unit 306 manages and processes data. In some embodiments, the processing unit 306 includes a digital core that digitizes analog signals transmitted from other components, performs computations, and executes algorithms to interpret the data from the biosensor 302 and the temperature sensor 304. In some examples, the processing unit 306 also manages data storage.

As previously discussed, the memory 308 stores data related to the operation of the biochip 300. In some embodiments, the memory 308 is a non-volatile memory that stores data permanently. For example, the memory 308 is a One-Time Programmable (OTP) memory that allows data to be written only once, after which the data is permanently stored and cannot be modified or erased. In some embodiments, the OTP memory 308 permanently stores calibration data or trimming coefficients or both, including temperature-based correction factors.

As shown in the example in FIG. 7, the memory 308 is a Triple Redundant OTP memory that surrounds the processing unit 306. The triple scheme ensures robustness and reliability by surviving (i.e., withstanding) any wafer-level or package-level processing damages. For example, even if the data in one or two OTP memories are corrupted or damaged during manufacturing, packaging, or operational stresses, the remaining OTP memory will retrieve the original data. The triple scheme is illustrated as an example, but the embodiment is not limited thereto. In other embodiments, the number of memories can be more or less than three, and the scheme can be different.

The data processing units 312a and 312b are signal processing components that reduce the high-frequency noise of the system and improve the signal quality. In some embodiments, the data processing units 312a and 312b reduce the high-frequency noise by downsampling the signal while preserving essential low-frequency components. For example, the first data processing unit 312a acts as a low-pass filter, removing high-frequency noise with frequencies above a certain cutoff threshold, thereby preventing aliasing when higher frequency signals are misinterpreted as lower frequency signals during the sampling process.

Once the higher frequencies have been filtered out by the first data processing unit 312a, the signal is downsampled. For example, downsampling involves reducing the overall number of samples in the signal by a specific factor, which lowers the data rate and simplifies further processing. The signal is passed to the second data processing unit 312b after downsampling. In some embodiments, the second data processing unit 312b further smooths the signal and refines noise suppression to reduce the residual noise not filtered by the first data processing unit 312a.

As shown in the example in FIG. 7, the first data processing unit 312a is positioned on the first side of the biosensor 302 to receive raw data from the first half of the biosensor 302, and the second data processing unit 312b is positioned on the second side of the biosensor 302 to receive raw data from the second half of the biosensor 302. In some embodiments, the first data processing unit 312a and the second data processing unit 312b convert the high-frequency data into a lower frequency stream, which is more manageable for subsequent digital processing stages.

The reference generator 314 is a component that provides reference signals or voltages to ensure the consistency in signal measurements. In some examples, the reference generator 314 generates reference signals necessary for accurate sensor compensation. For example, the reference generator 314 adjusts the reference signals to compensate for environmental variations such as temperature fluctuations, ensuring consistent and precise compensation under various conditions.

The test control 316 is a dedicated area designed to monitor the performance of the assay, acting as an internal quality control. In some embodiments, the test control 316 provides a baseline signal to compare against the test result from the sample to ensure the validity and reliability of assay results. In some examples, the test control 316 detects errors or anomalies during the testing process.

The resistive heater 318 is a microscale heating element that generates heat when an electrical current passes through the resistive material. In some embodiments, the resistive heater 318 adjusts the temperature of the biochip 300 to accelerate biomedical reactions.

The I/O pads 322 are conductive elements designed to connect to an external circuit. In some embodiments, the I/O pads 322 connect to an electrical circuit to facilitate signal amplification, processing, and transmission. In some examples, the electrical circuit converts the biochemical signals into readable electrical outputs that will be analyzed further.

Any of the memory described herein can be implemented as computer readable media. Computer readable media includes any available media that can be accessed by the processing unit 306. By way of example, computer readable media include computer readable storage media and computer readable communication media.

Computer readable storage media includes volatile and nonvolatile, removable, and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the processing unit 306. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

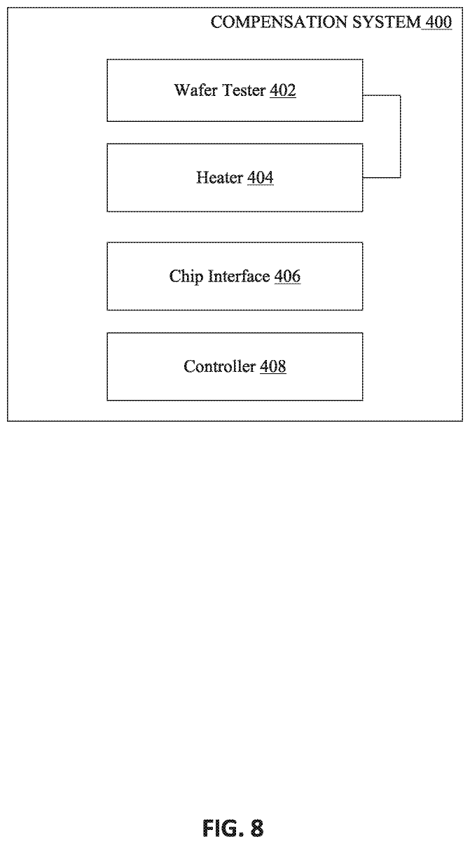

FIG. 8 is a block diagram illustrating an example compensation system 400. The example compensation system 400 includes a wafer tester 402, a heater 404, a chip interface 406 and a controller 408.

The compensation system 400 is designed to calibrate the biochip, trim the biochip, or a combination thereof during the manufacturing process. In some embodiments, the compensation system 400 interacts with the biochip to ensure that the fabricated biochip meets expected performance specifications by verifying and adjusting various parameters during production and testing.

The wafer tester 402 is a component used to test the electrical properties of semiconductor wafers. In some embodiments, the wafer tester 402 is designed to interface with the wafer directly. For example, the wafer tester 402 interacts with the wafer to evaluate the functionality and quality of semiconductor devices by applying electrical signals to the chips on the wafer and measuring the output. In some examples, the wafer tester 402 is not only configured to execute the compensation process but also integrates the heater 404 to regulate the temperature of the wafer during compensation, ensuring that the temperature conditions are optimal for accurate testing.

The heater 404 is configured to maintain a controlled temperature environment for the wafer. In some embodiments, the heater 404 is integrated into the wafer tester 402 and designed to bring the wafer to a specific temperature during the compensation process. For example, because the properties of the semiconductor materials are affected by the temperature, maintaining a controlled thermal environment ensures accurate and reliable electrical measurements.

The chip interface 406 establishes both the physical and electrical connection between the wafer tester 402 and the biochip on the wafer. In some embodiments, the chip interface 406 serves as a signal transmission interface, facilitating the exchange of test signals between the wafer tester 402 and the biochip. For example, this interface enables measurement and analysis of the electrical characteristics of the biochip, ensuring proper functionality and compensation. In some examples, depending on the testing requirements, the chip interface 406 may include probe contacts, microelectrode arrays, or other specialized connectors designed to minimize signal distortion and enhance test accuracy.

The controller 408 is the central unit that coordinates the activities of the compensation system 400. In some embodiments, the controller 408 manages the operation of the wafer tester 402, the heater 404, and the monitoring of the chip interface 406. For example, the controller executes software that processes input from these components and dynamically adjusts their operation to maintain precise compensation. In some examples, the controller 408 functions as the command center of the compensation system 400, overseeing the operation of the integrated heater 404. It utilizes real-time feedback from temperature sensors on the biochip to dynamically adjust the heater settings, ensuring that the wafer remains at optimal conditions for accurate compensation. This closed-loop temperature control minimizes fluctuations and enhances the reliability of the compensation process.

In some embodiments, the controller 408 manages the operation of the heater, determine the temperature-based correction factor, and provide the temperature-based correction factor to the biochip. In some examples, the controller 408 adjusts the temperature-based correction factor based on a comparison between the temperature reading of the biochip and a reference temperature value.

FIG. 9 is a flow chart illustrating an example method 420 of a wafer-level compensation process for biochips. The example method 420 includes operations 422, 424, 426, 428, 430, 432, and 434. Other embodiments include more, fewer, or different operations from the example illustrated in FIG. 9.

The operation 422 is performed to prepare a calibration instrument. In some embodiments, the calibration instrument is set up and verified for accuracy. In some examples, the calibration instrument is configured to apply controlled temperature variations and record sensor readings. Further, necessary software scripts or automated calibration procedures are loaded into the system of the calibration instrument.

The operation 424 is performed to set up wafer temperature. For example, the entire wafer is placed in a temperature-controlled chamber or on a heated chuck to simulate real-world operating conditions. In some embodiments, the temperature is precisely controlled within a tight tolerance (e.g., ±0.1 °C) to ensure repeatable calibration. However, in other embodiments, the tolerance is not strictly constrained and may vary based on specific calibration requirements. In some examples, a reference temperature sensor in the operation 424 is applied to verify the actual temperature of the wafer.

The operation 426 is performed to probe all of the biochips on the wafer. In some embodiments, wafer probes are used to make electrical contact with each biochip on the wafer. For example, the wafer probes are fine-tipped probes that touch specific contact pads of each biochip to supply power and read the temperature sensor outputs. In some examples, sensor readings from the temperature sensor are recorded at various temperatures to determine how each chip responds.

The operation 428 is performed to trim the temperature sensor. In some embodiments, the trimming process ensures that each sensor provides an accurate and consistent temperature reading across the wafer. For example, the temperature sensor trimming process compensates for variations in fabrication, material properties, and circuit characteristics. In some examples, the trimming process also includes compensation for environmental factors. For example, multi-temperature trimming is performed to adjust the sensor response at different temperature points to ensure accuracy across a broad range. In some embodiments, the trimming process calculates correction values based on deviations between measured sensor output and the expected reference values.

The operation 430 is performed to store trim coefficients or calibration data in a memory. In some embodiments, once the appropriate corrections or data are determined, trim coefficients or calibration data are stored in the memory of each biochip. For example, the coefficients or calibration data are stored for use in real-time by the biochip during normal operation to adjust raw sensor readings and improve accuracy. In some examples, multiple trim coefficients or calibration data are stored to correct for different temperature ranges.

The operation 432 is performed to complete the trimming and storage process for all of the biochips on the wafer. For example, the trimming and coefficient storage process is repeated for every biochip on the wafer. In some embodiments, the operation 432 is performed to ensure each biochip meets required performance specifications. In some examples, the defective biochips that fail calibration will be removed.

The operation 434 is performed to store the wafer for future use. In some embodiments, the wafer is stored under controlled conditions to maintain its accuracy and reliability after compensation. For example, the wafer is packaged in a protective enclosure to prevent contamination. In some examples, the wafer is sent for dicing where individual biochips are cut from the wafer and packaged.

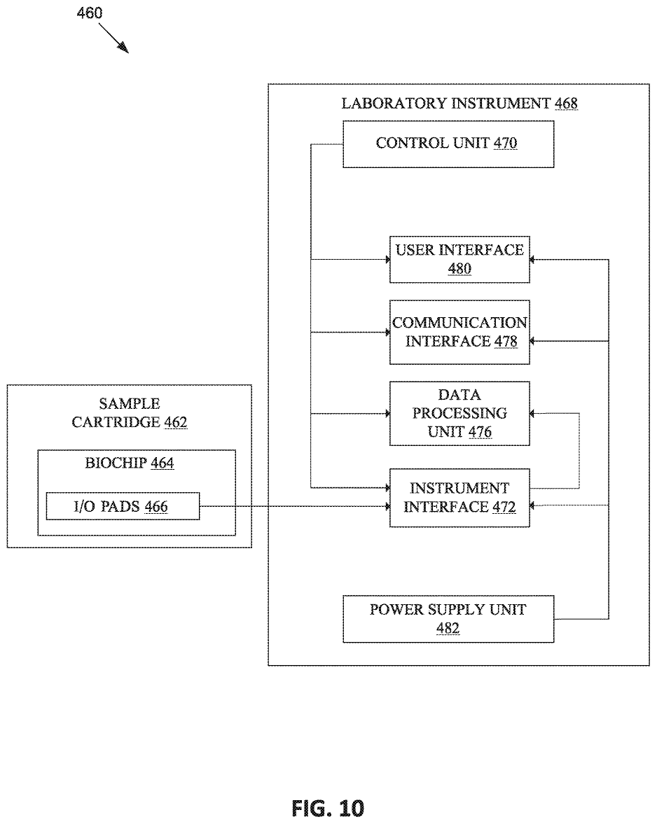

FIG. 10 is a block diagram illustrating an example testing system 460 that includes a sample cartridge 462 and a laboratory instrument 468. In the example testing system 460, the sample cartridge 462 includes a biochip 464 with a plurality of I/O pads 466. The example testing system 460 also includes a laboratory instrument 468. As an example illustrated in FIG. 10, the laboratory instrument 468 includes a control unit 470, an instrument interface 472, a data processing unit 476, a communication interface 478, a user interface 480, and a power supply unit 482.

The example testing system 460 is configured to analyze a biological sample. In some embodiments, the laboratory instrument 468 is designed to interface with the sample cartridge 462 through an instrument interface, facilitating electrical and data communication.

As previously discussed, the sample cartridge 462 is designed for receiving and processing a biological sample for analysis by the laboratory instrument 468. In some embodiments, the sample cartridge 462 is inserted into the laboratory instrument 468 and interfaced with the instrument interface 472 for analytical processing.

As previously discussed, the biochip 464 is a microelectronic device that includes sensors or detection elements for analyzing the biological sample. In some embodiments, the biochip 464 utilizes electrochemical, optical, or impedance-based sensing techniques to detect target analytes such as nucleic acids, biomarkers, proteins, metabolites, or other biologically significant molecules. These target analytes are obtained, isolated, collected, or captured from a sample, including, without limitation, blood, biological fluids, liquefied solids, or tissue. The process enables the biochip 464 to detect and analyze specific biological markers, facilitating diagnostic or research applications.

As previously discussed, the I/O pads 466 are conductive elements integrated into the biochip 464. In some embodiments, the I/O pads 466 function as the sensing interface, facilitating electrochemical reactions, signal transduction, or impedance measurement to generate raw data for processing. In some examples, the I/O pads 466 are connected to the instrument interface 472, allowing the sample cartridge 462 to interact with the laboratory instrument 468 for data acquisition and analysis.

As previously discussed, the laboratory instrument 468 is a processing and analysis device configured to receive the sample cartridge 462. In some embodiments, the laboratory instrument 468 interacts with the sample cartridge 462 to facilitate the sample data detection, measurement, and processing. In some examples, the laboratory instrument 468 analyzes the data received from the biochip 464 and provides test results to the user.

The control unit 470 is the central processing unit of the laboratory instrument 468. In some embodiments, the control unit 470 manages the overall operation of the laboratory instrument 468. For example, the control unit 470 controls interactions between different internal components and regulates measurement cycles, compensation processes, and data processing tasks. In some examples, the control unit 470 is implemented as a microcontroller, microprocessor, or embedded computing system with dedicated firmware. In some embodiments, the control unit 470 sends instructions to internal components of the laboratory instrument 468 or to external devices via signals transmitted through the communication interface 478.

As previously discussed, the instrument interface 472 serves as the primary connection between the laboratory instrument 468 and the sample cartridge 462. In some embodiments, the instrument interface 472 provides electrical connectivity to power the biochip 464 and I/O pads 466. In some examples, the pogo pins of the instrument interface 472 connect to the I/O pads 464 of the biochip, allowing the instrument interface 472 to receive raw signals from the biochip 464. In some embodiments, the signals received from the biochip 464 are transmitted to the data processing unit 476 through the instrument interface 472.

The data processing unit 476 is configured to analyze and process the acquired data and signals. In some embodiments, the data processing unit 476 analyzes and interprets the signals received from the biochip 464 to extract useful information. For example, the data processing unit 476 interprets data by applying algorithms for pattern recognition, statistical analysis, or diagnostic evaluation. Further, the data processing unit 476 converts the analog signals into digital data. In some examples, the data processing unit 476 performs signal amplification and filtering by enhancing weak signals and removing unwanted noise.

The communication interface 478 is an interface, such as a communication media, between the laboratory instrument 468 and external devices. In some embodiments, the communication interface 478 enables data exchange between the laboratory instrument 468 and external devices by wired communication (e.g., USB, Ethernet, or serial data transfer) or wireless communication (e.g., Bluetooth, Wi-Fi, or NFC for remote data access).

The user interface 480 provides an interactive platform for users to operate and monitor the laboratory instrument 468. In some embodiments, the user interface 480 includes a touchscreen display or LCD screen to present real-time results and system status. In some examples, the user interface 480 includes physical or virtual buttons for navigation and command input. In some embodiments, the user interface 480 enables the user to interact with the laboratory instrument 468 to initiate tests, review analysis results, and configure system settings.

The power supply unit 482 provides the necessary electrical energy for all internal components of the laboratory instrument 468. In some embodiments, the power supply unit 482 connects to each component of the laboratory instrument 468 to supply the power to ensure stable operations.

FIG. 11 is a flow chart illustrating an example process 500 for performing measurements using a biochip within an instrument. The example process 500 performs the operations 502, 504, 506, 508, 510, 512, 514, 516, and 520. Other embodiments include more, fewer, or different operations from the example illustrated in FIG. 11.

The operation 502 is performed to power on the instrument. In some embodiments, the instrument is powered on once the cartridge is loaded, initiating the internal processes required for measurement.

The operation 504 is performed to load the cartridge into the instrument. In some embodiments, the user inserts the cartridge, which includes the biochip and necessary reagents, into the instrument.

The operation 506 is performed to read trim coefficients from the memory. In some embodiments, the instrument retrieves pre-stored trim coefficients from the memory to compensate for variations in sensor readings. For example, the retrieved coefficients adjust measurements for temperature, voltage offsets, fabrication differences and other variations to improve accuracy.

The operation 508 is performed to set up the biochip. In some embodiments, the biochip is initialized for operation. For example, the operation 508 includes applying power and initializing its circuits, setting up internal parameters (e.g., voltage levels, reference currents), and preparing fluidic pathways for interaction with the sample.

The operation 510 is performed to run the test. In some embodiments, the testing process begins based on predefined protocols. For example, the biochip operates according to its designed function, which includes biochemical, electrical, optical, or other applicable measurements.

The first sub-step 512 of the operation 510 is performed to start the measurement process. In some embodiments, the instrument begins the measurement sequence at the first sub-step 512, preparing to interact with the sample through the biochip.

The second sub-step 514 of the operation 510 is performed to cause the biochip to interact with the sample under test. In some embodiments, the biochip is exposed to the sample at the second sub-step 514, initiating a reaction or interaction that generates a measurable signal.

The operation 516 is performed to collect and process data from the biochip. In some embodiments, the data processing unit collects and processes data produced by the biochip as it interacts with the sample at the operation 516. In some examples, the collected data includes electrical, optical, or biochemical signals. Further, the data will be temporarily stored in the data processing unit for additional analysis.

The operation 520 is performed to display the results to the user. In some embodiments, the final measurement results are displayed to the user in a reasonable format, providing diagnostic information. For example, the format includes graphical representations such as charts or graphs and numeric data displayed in tables. In some examples, color coding or other visual aids are used to highlight critical values or trends, making it easier for users to assess the diagnostic information provided quickly.

The various embodiments described above are provided by way of illustration only and should not be construed to limit the claims attached hereto. Those skilled in the art will readily recognize various modifications and changes that may be made without following the example embodiments and applications illustrated and described herein, and without departing from the full scope of the following claims.

1. A biochip comprising:
a biosensor configured to detect target analytes;
a temperature sensor configured to provide temperature data for compensating the biosensor;
a processing unit configured to process signals from the biosensor and the temperature sensor; and
a memory configured to store calibration data, trim coefficients, or both.

2. The biochip of claim 1, wherein the calibration data, the trim coefficients, or both include a temperature-based correction factor.

3. (canceled)

4. The biochip of claim 1, wherein the memory includes at least two memory cells.

5. (canceled)

6. The biochip of claim 1, wherein the memory is at least three memory cells.

7. (canceled)

8. The biochip of claim 1, wherein at least one of the biosensor, the temperature sensor, the processing unit, and the memory is integrated on a single chip.

9. The biochip of claim 8, wherein all of the biosensor, the temperature sensor, the processing unit, and the memory are integrated on the single chip.

10. The biochip of claim 1, wherein the biosensor, the temperature sensor, the processing unit, and the memory are integrated on a single chip by using a complementary metal-oxide-semiconductor (CMOS) technology.

11. The biochip of claim 1, wherein the temperature sensor is proximal to the biosensor.

12. The biochip of claim 1, further comprising a pair of data processing units that are positioned on opposite sides of the biosensor to reduce high-frequency noise.

13. (canceled)

14. The biochip of claim 1, further comprising a heater that adjusts the temperature of the biochip.

15. (canceled)

16. (canceled)

17. The biochip of claim 14, wherein the heater is a resistive heater.

18. (canceled)

19. A system configured to perform compensation, the system comprising:
a wafer tester configured to interface with a wafer and execute a compensation process;
a heater configured to regulate the temperature of the wafer during the compensation process;
a chip interface configured to facilitate data communication with a biochip for obtaining a temperature reading of the biochip, wherein the biochip includes a temperature sensor configured to provide temperature data for compensation, and a memory configured to store calibration data, trim coefficients, or both, including a temperature-based correction factor; and
a controller configured to manage the operation of the heater, and determine the temperature-based correction factor.

20. (canceled)

21. The system of claim 19, wherein the controller adjusts the temperature-based correction factor based on a comparison between the temperature reading of the biochip and a reference temperature value.

22. (canceled)

23. A method for calculating and storing calibration data, trimming coefficients, or both in biochips of a wafer, the method comprising:
receiving temperature data from a temperature sensor of the biochips during a compensation process;
regulating the temperature of the wafer by controlling a heater based on the temperature data to achieve a predetermined condition;
measuring operating parameters of the biochips under the predetermined condition;
calculating calibration data, trim coefficients, or both, including temperature-based correction data, based on the measured operational parameters and the temperature data; and
storing the calibration data, trim coefficients, or both in a memory of each biochip.

24. The method of claim 23, further comprises measuring the temperature data by the temperature sensor.

25. The method of claim 23, further comprises recording the temperature data in the memory.

26. The method of claim 23, further comprises programming the memory to store the calibration data, trim coefficients, or both.

27-29. (canceled)

30. The method of claim 23, wherein the memory comprises a triple redundant memory.

31. The method of claim 30, wherein the calibration data, the trim coefficients, or both are stored in the triple redundant memory.

32. The method of claim 23, wherein the calibration data, the trim coefficients, or both are permanently stored in the memory.

33-68. (canceled)
